(19) United States
(12) Patent Application Publication
(10) Pub. No.: US 2026/0137539 A1
(43) Pub. Date: May 21, 2026

(54) Medical Cast Made Out of Many Hinged Sections

(71) Applicants: Corban Walsh, Billerica, MA (US); Kaitlyn Shelver, Birmingham, AL (US); Ashleigh Woods, Manchester, NH (US)

(72) Inventors: Corban Walsh, Billerica, MA (US); Kaitlyn Shelver, Birmingham, AL (US); Ashleigh Woods, Manchester, NH (US)

(21) Appl. No.: 18/949,879

(22) Filed: Nov. 15, 2024

(51) Int. Cl. A61F 5/01

(52) U.S. Cl. CPC A61F 5/0104

(57) ABSTRACT

A medical cast that is breathable, hygienic, and reusable with many hinged sections. The medical cast allows for a more convenient and comfortable healing process for injured patients. The cast has better air flow and adjustable straps.

### PRIOR ART

The present disclosure relates, in general, to medical casts with the purpose of being reusable, breathable, more hygienic, and more convenient.

EP 2359288 B1 discloses a cast that uses hinges as part of its cast design.

US 2022/0249267 A1 discloses a medical device that incorporates straps with its brace system.

WO 2014/082502 A1 discloses a medical cast with air intake and increased breathability.

WO 2008/157738 A1 discloses a cast system composed of thermoformable structural material such as perforated plastic.

However, there is currently not a medical cast that relies on hinges that rotate on only one axis and that has multiple flexible strands that can cater to different patients. Therefore, a need exists for this flexible and reusable cast.

### BACKGROUND OF THE INVENTION

The present subject matter is a medical cast that is composed of many small plastic segments, allowing it to fit different patients and be reusable.

### SUMMARY OF THE INVENTION

The present subject matter is a medical cast that allows an injury to heal, while being reusable, breathable, more hygienic, and more convenient.

The present subject matter relies on the properties of hinges.

The present subject matter has many small plastic segments, with a hinge at two opposite ends such that they can be attached and made into a flexible strand of segments.

The present subject matter has flexible strands which can fit many different patients and then be made rigid. This allows the present subject matter to be reusable because it can be used by different patients.

The present subject matter is more hygienic because it is entirely made of plastic, allowing it to be easily cleaned.

The present subject matter is breathable because it is a non-continuous structure.

The foregoing summary is illustrative only and is not intended to be in any way limiting. In addition to the illustrative aspects, aspects, and features described above, further aspects, aspects, and features will become apparent by reference to the drawings and the following detailed description.

Other systems, methods, features, and advantages of the disclosure will be, or will become, apparent to one with skill in the art upon examination of the following figures and detailed description. It is intended that all such additional systems, methods, features and advantages be included within this description, be within the scope of the disclosure, and be protected by the following claims.

### BRIEF DESCRIPTION OF THE DRAWINGS

The disclosure can be better understood with reference to the following drawings and description of an aspect. The elements in the figures are not necessarily to scale, emphasis instead being placed upon illustrating the principles of an aspect of the disclosure. Moreover, in the figures, like referenced numerals designate corresponding parts throughout the different views of the aspect.

FIG. 1 illustrates a first view of a hinge for a medical cast according to an aspect of the disclosure.

FIG. 2 illustrates a second view of a hinge for a medical cast according to an aspect of the disclosure.

FIG. 3 illustrates a side view of an example medical cast according to an aspect of the disclosure.

### DETAILED DESCRIPTION

It is imperative to create comfortable, breathable, clean, and reusable medical casts to allow injuries to heal in the most convenient way possible.

The disclosed medical cast is entirely made of plastic, allowing it to be cleaned easily.

This way, people do not need to purchase a new cast when it gets dirty.

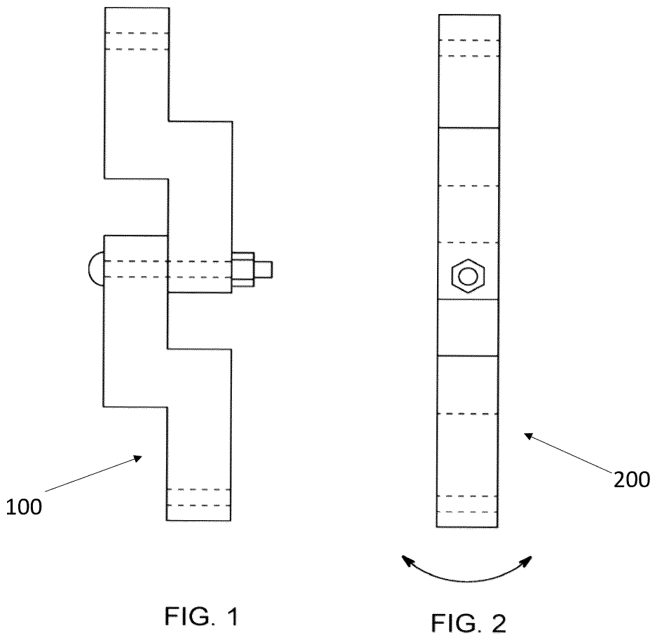

FIG. 1 and FIG. 2 illustrate views of an exemplary hinge (100, 200) that may be used in the disclosed medical cast. The disclosed medical cast relies on the properties of hinges. These hinges (100, 200) rotate on only one axis.

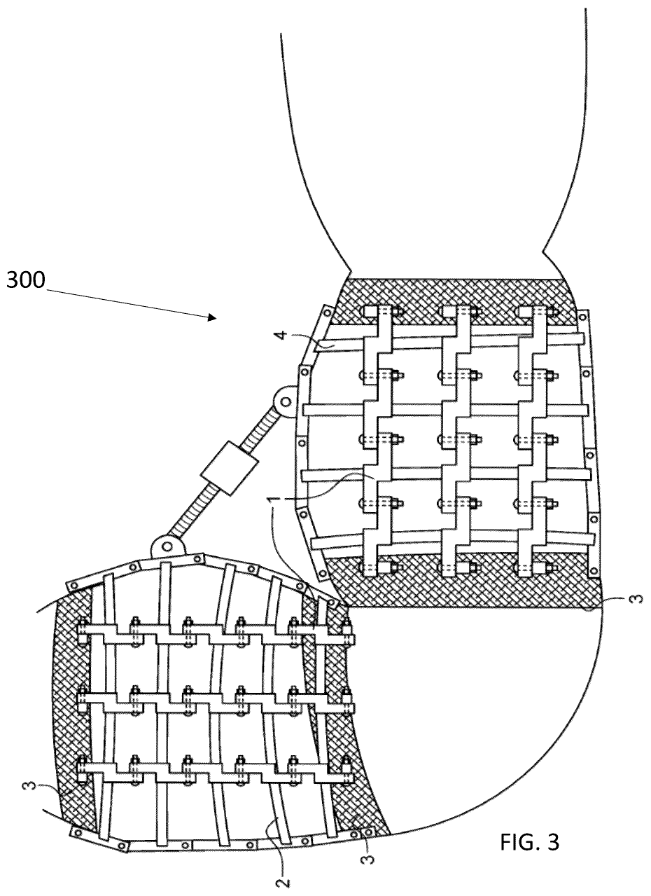

FIG. 3 side view of an example medical cast 300 according to an aspect of the disclosure. The cast 300 is composed of many small plastic segments 1, with a hinge at two opposite ends. This allows them to attach to each other and be made into a flexible strand 2, 4 of segments. These strands 2, 4 are placed around the circumference of the injured body part. If the limb were to bend, some hinges would be aligned correctly, but many would not, impeding motion.

The strands are secured with a strap 3. Because the strands are flexible, they can fit many different patients, meaning the cast 300 can be reusable. The disclosed cast 300 can be reassembled to fit vastly different injuries on different patients because the segments can be easily added or subtracted, and the strands can be formed to the body.

The disclosed cast has a non-continuous structure. This allows for air flow, making the cast more breathable.

The disclosed cast achieves rigidity through geometry instead of a brute force method.

Many modifications and other aspects of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. Furthermore, in some aspects, additional optional operations may be included. Modifications, additions, or amplifications to the operations above may be performed in any order and in any combination.

Moreover, although the foregoing descriptions and the associated drawings describe example aspects in the context of certain example combinations of elements and/or functions, it should be appreciated that different combinations of elements and/or functions may be provided by alternative aspects without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A medical cast, that is adaptable to different patients and injuries comprising:
a. Hinges that rotate on only one axis;
b. Many small plastic segments;
c. Flexible stands that can fit different patients, and then be made rigid; and
d. Entirely plastic in composition.

2. The medical cast of claim 1, wherein the small plastic segments allow for adaptability and reusability.

3. The medical cast of claim 1, wherein the flexible strands can be easily formed to different bodies and limbs.

4. The medical cast of claim 1, wherein there are hinges that rotate on only one axis.
